(19) United States
(12) Patent Application Publication
(10) Pub. No.: US 2022/0202375 A1
(43) Pub. Date: Jun. 30, 2022

(54) WEARABLE MEASUREMENT MANAGEMENT

(71) Applicant: International Business Machines Corporation, Armonk, NY (US)

(72) Inventors: Zachary A. Silverstein, Austin, TX (US); Jeremy R. Fox, Georgetown, TX (US); Sarbajit K. Rakshit, Kolkata (IN); Uri Kartoun, Cambridge, MA (US)

(21) Appl. No.: 17/139,094

(22) Filed: Dec. 31, 2020

(51) Int. Cl. A61B 5/00; G16H 40/67; G16H 20/30

(52) U.S. Cl. CPC A61B 5/7285; A61B 5/7221; A61B 5/6802; A61B 5/02438; G16H 20/30; A61B 2560/0209; A61B 5/0024; G16H 40/67

(57) ABSTRACT

First and second wearable devices are determined to both be worn by a user during a first activity and are both configured to measure a same condition relating to the user. Based on how characteristics of the first and second wearable devices relate to the first activity, it is determined to measure the same condition with only the first wearable device. The first and second wearable devices are determined to both be worn by the user during a second activity. Based on how characteristics of the first and second wearable devices relate to the second activity, it is determined to measure the same condition with only the second wearable device.

### BACKGROUND

The Internet of Things (IoT) wearable market is growing at an increasingly fast rate. Sensors configured to sense and communicate various immediate and/or ongoing conditions relating to a user are being integrated into shoes, shirts, gloves, glasses, watches, and other devices that can be worn or otherwise brought with users throughout their daily lives. Often times many of these wearable devices can have overlapping functionality. For example, a user may have two or three wearable devices that detect heart rate, two or three different wearable devices that detect steps/paces, or the like.

### SUMMARY

Aspects of the present disclosure relate to a method, system, and computer program product relating to management measurements gathered by wearable devices. For example, the method includes determining that a first wearable device and a second wearable device are both worn by a user during a first activity of the user and are both configured to measure a same condition of the user. The method also includes determining, based on how characteristics of the first and second wearable devices relate to the first activity, to measure the same condition with only the first wearable device. The method also includes determining that the first and second wearable devices are both worn by the user during a second activity. The method also includes determining, based on how the characteristics of the first and second wearable devices relate to the second activity, to measure the same condition with only the second wearable device. A system and computer product configured to perform the above method are also disclosed.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a conceptual diagram of an example system in which a controller may manage measurements of conditions related to a user as gathered by a plurality of wearable devices worn by a user.

FIG. 2 depicts a conceptual box diagram of example components of the controller of FIG. 1.

FIG. 3 depicts an example flowchart by which the controller of FIG. 1 may manage measurements of conditions related to a user as captured by wearable devices.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to managing measurements of wearable devices, while more particular aspects of the present disclosure relate to dynamically modifying what wearable devices are measuring what conditions of a user at different times depending on different characteristics of the wearable devices and different activities of the user. While the present disclosure is not necessarily limited to such applications, various aspects of the disclosure may be appreciated through a discussion of various examples using this context.

The proliferation of wearable devices enables users to gather a substantial amount of information about themselves and the world around them. For example, a user may own different wearable devices that include sensors that are configured to sense or measure conditions relating to the health of the user, the activity of the user, the sleep of the user, the environment of the user, and the like. A user may own a substantial set of wearable devices that may be worn or otherwise secured to the user in different ways and at different times. For example, a user may utilize such wearable devices as a smart watch, smart glasses, smart earbuds, a heart monitor, a smart wristband or anklet, or other such computing devices that may be removably secured to a body of the user. A user may also have wearable devices that are within clothing of a user, such as smart shoes, a smart hat, smart gloves, or other such clothing articles that may contain one or more sensors that may be positioned near and/or against a body of a user to gather various conditions relating to the user. In some examples, wearable devices may include computing components that are temporarily or permanently secured within or to a user, such as a smart tattoo, an ingestible sensor, an implanted sensor (such as an implantable medical device), or the like. As used herein, any of these devices may be described as a wearable device.

These wearable devices may include one or more sensors that are configured to sense or measure one or more conditions associated with a user. For example, these wearable devices may include sensors configured to measure conditions related to a body of the user, such as footsteps (e.g., foot strikes) of a user, a mileage traveled by the user, a heart rate of the user, a blood pressure of the user, a location (e.g., such as measured with global positioning system (GPS) coordinates) of the user, a temperature of the user, a sleep state (e.g., whether sleeping in general, in rapid eye movement (REM) sleep, deep sleep, light sleep) of the user, or the like. Further, wearable devices may also be configured to measure conditions related to an environment of the user, such as a visual environment of a user (e.g., pictures or a video feed around the user), an audible environment of a user, a temperature of the air around the user, or the like.

In some examples, two (or more) wearable devices may be configured to measure a same condition, such that the same information is gathered twice. Further, some of these wearable devices may measure these conditions in different ways. Some may measure them with greater granularity, while others measure them with more efficiency, while others measure them more reliability, etc. In many examples, each of these wearable devices must be powered by an individual power supply.

As such, in a conventional system, two or three or more wearable devices may be using respective finite power supplies to gather redundant data in the form of two (or more) measurements for the same condition. For example, if two wearable devices are measuring a same condition, one of these two wearable devices may run out of power faster than the other. In a conventional system, a user may try to individually choose which wearable device will measure which condition. This process may be error-prone and time-intensive, as a user may be required to select a new wearable device to identify a condition when the initial wearable device is low on battery power or the like. Further, on top of being a cumbersome solution, it may be difficult or impossible for a user to identify when one wearable device is relatively more efficient, reliable, accurate, or the like to measure one or more conditions for different activities (and therein also to again remember to switch between these wearable devices during different conditions).

Further, even if a user is capable of identifying a solution as to which of a current set of worn wearable devices should be measuring which condition for a current activity, some users may regularly put on or take off different wearable devices on different days and/or at different hours of the day. This mean that a previous “solution” as identified by a user may be impossible (e.g., such that the selected wearable device is no longer being worn), improper (such that the selected wearable device cannot gather this condition in the current activity, such as a wristband wearable device configured to measure distance traveled through a gyroscope while a user is biking), or relatively inefficient as discussed herein for a current set of worn wearable devices. For example, a user may select a pair of workout shoes as the only wearable device to measure a distance traveled condition, after which a user may finish the workout and swap the workout shoes for work shoes, therein going to work. In this example, absent the user remembering and reconfiguring the full set of wearable devices, this previous solution (to have the workout shoes measure the distance traveled condition) may result in this condition not being measured while the user is at work.

Aspects of this disclosure are related to solving these technical problems created by having a plurality of wearable devices that can all measure an overlapping array of conditions associated with a user. For example, aspects of the disclosure relate to identifying all wearable devices associated with a user, determining which wearable devices are currently being worn, and then determining which wearable devices should measure which conditions based on an activity of a user and various characteristics of the wearable devices. A computing device that includes a processing unit executing instructions stored on a memory may provide this functionality, and this computing device is herein referred to as a controller. This controller may be contained within one of the wearable devices, within a cell phone of a user, or within a separate device (e.g., within a hosting server that communicates with the wearable devices over a network).

The controller may gather information on all of the wearable devices. The controller may gather this information by having all of the wearable devices measure all conditions which the respective wearable devices are configured to measure during an initial session. The controller may compare these conditions against each other, and also compare how efficient each wearable device was at measuring each condition (e.g., whether some wearable devices were able to measure some conditions at a rate that consumed more or less relative battery power than other wearable devices). The controller may then run an analysis using all of these measured conditions to determine characteristics of various wearable devices. These determined characteristics may reflect which wearable devices are better for measuring which conditions for which activities. Once all of this data is gathered and all of these characteristics are determined, the controller may then dynamically manage which wearable device is measuring which condition related to a user based on changing sets of wearable devices worn by the user, changing activities of the user, changing characteristics of the wearable devices, or the like. For example, during a first session of a first activity the controller may cause a first wearable device to measure a condition, but during a subsequent session of this first activity the controller may cause a second wearable device to measure the condition. The change to the second wearable device may be based on a determination that the first wearable device is low on power, a determination that the first wearable device has been taken off by the user, a determination that the first wearable device has been taking inaccurate measurements, or the like.

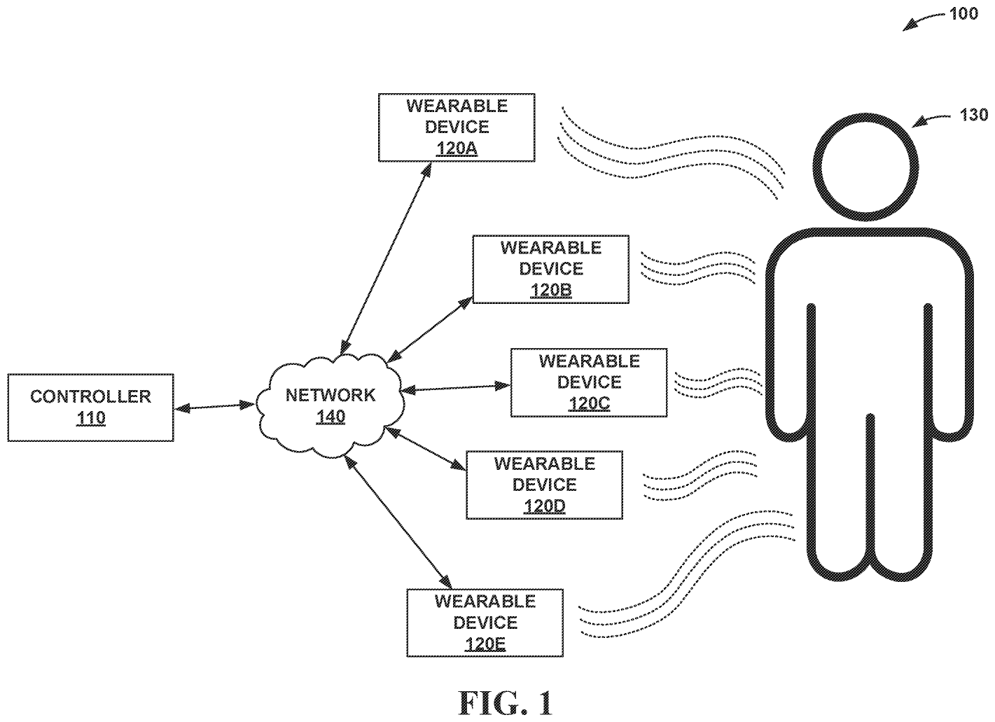

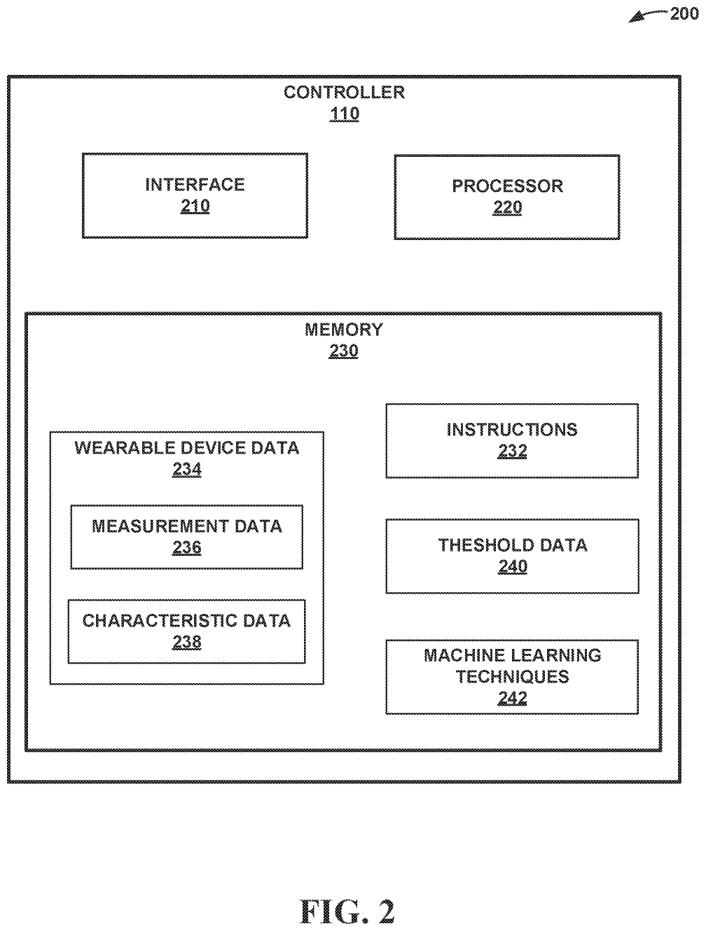

For example, FIG. 1 depicts an environment 100 in which the controller 110 manages measurements gathered by a plurality of wearable devices 120A-120E (collectively “wearable devices 120). Controller 110 may include a computing device, such as computing system 200 of FIG. 2, that includes a processor communicatively coupled to a memory that includes instructions that, when executed by the processor, causes controller 110 to execute one or more operations described below. In some examples, controller 110 may be a user device that controls and/or receives data from one or more wearable devices 120. For example, controller 110 may be a cell phone of user 130.

As described above, each of wearable devices 120 may be worn by or otherwise secured to user 130. Wearable devices 120 may include at least one sensor configured to sense or measure at least one condition relating to user 130. For example, wearable device 120A may be smart glasses configured to measure audible and visual conditions around user 130 (e.g., such that smart glasses wearable device 120A is configured to store visual conditions of where user 130 was looking and audible conditions of sounds that user 130 heard) and a GPS condition of user 130. For another example, wearable device 120B may include a heart rate monitor that is configured to measure a heart rate condition of user 130, a run cadence condition of user 130, and a stride length condition of user 130. Further, wearable device 120C may be a smart watch configured to measure footsteps and GPS conditions of user 130, wearable device 120D includes pants that are embedded with sensors configured to identify leg movement conditions of user 130 (e.g., when the legs of user 130 are moving, how fast the legs are moving, etc.), while wearable device 120E may include smart shoes configured to measure footsteps of user 130.

In some examples, before interacting with any wearable devices 120 or executing any other operations described herein, controller 110 may be configured to request an express “opt-in” from user 130. For example, this opt-in may include expressly list all data that controller 110 may access, all purposes for which controller 110 may gather this data, and all functionalities which controller 110 is configured to execute. In some examples, controller 110 may gather this opt-in repeatedly on a schedule (e.g., once every year, or once every six months), to give user 130 an ability to change settings of controller 110 as desired. Controller 110 may only manage wearable device condition measurement in response to receiving such opt-ins from user 130.

Controller 110 may determine what conditions each of wearable devices 120 can measure. Specifically, controller 110 may determine if two or more of wearable devices 120 are configured to detect the same condition. For example, as described above, controller 110 may identify that both wearable device 120A and 120C are configured to measure a GPS condition, and both wearable device 120C and wearable device 120E are configured to measure a footsteps condition.

When controller 110 identifies that two or more wearable devices 120 measure duplicate data for a same condition, controller 110 may cause all of these wearable devices 120 to measure the duplicate data at the same time so that the data may be cross-compared. By comparing data measured by one wearable device 120 for a condition during an activity against other sets of data for other conditions during that same activity, so that controller 110 may identify which wearable device 120 measures condition data in what way for what activity. In this way, controller 110 may determine characteristics of each wearable device 120.

For example, controller 110 may determine a frequency with which each wearable device 120 can measure conditions, an accuracy of measured conditions, a repeatability of measured conditions, or the like. For example, controller 110 may determine that wearable device 120C measures footsteps more efficiently (e.g., taking less relative battery life), while wearable device 120E measures footsteps more accurately (e.g., not mistaking lifting a weight with an arm as a footstep). As such, controller 110 may cause wearable device 120C to measure footsteps during those times when footsteps are less volatile (e.g., when user 130 is at work) and cause wearable device 120E to measure footsteps during those times when footsteps require more accuracy to properly detect (e.g., when user 130 is working out).

Controller 110 may also be regularly checking which wearable device 120 is being worn by user 130 (or is otherwise near enough to user 130 to measure conditions related to user 130). For example, controller 110 may attempt to verify that a computing signal (e.g., a radio frequency identification (RFID) signal or a ZigBee signal or the like) of each wearable device 120 is detectable by a master device (e.g., a cell phone of user 130, which in some examples may also be included herein as one of wearable devices 120). In other examples, controller 110 may gather data from all wearable devices 120 that are turned on, with the assumption that wearable devices 120 are turned on when they are worn by user 130. In some examples, controller 110 may determine that wearable devices 120 are turned on when a location of wearable devices 120 changes, and/or when an internal gyroscope or the like of wearable devices 120 indicates that wearable devices 120 are moving.

Controller 110 may interact with wearable devices 120 over network 140. Network 140 may include a computing network over which computing messages may be sent and/or received. For example, network 140 may include the Internet, a local area network (LAN), a wide area network (WAN), a wireless network such as a wireless LAN (WLAN), a short-range communication technology such as Bluetooth (e.g., which may enable controller 110 to directly connect with each of wearable devices 120), a low power WAN (LPWAN) such as a 5G cellular network, or the like. Network 140 may comprise copper transmission cables, optical transmission fibers, wireless transmission, routers, firewalls, switches, gateway computers and/or edge servers. A network adapter card or network interface in each computing/processing device (e.g., controller 110 and/or wearable devices 120) may receive messages and/or instructions from and/or through network 140 and forward the messages and/or instructions for storage or execution or the like to a respective memory or processor of the respective computing/processing device. Though network 140 is depicted as a single entity in FIG. 1A for purposes of illustration, in other examples network 140 may include a plurality of private and/or public networks over which controller 110 may monitor conversations.

Using network 140, controller 110 is configured to dynamically re-configure which wearable devices 120 is capturing which condition to improve an ability of wearable devices 120 to capture a health and activity state of user 130. For example, controller 110 may verify an appropriate frequency with which certain conditions should be measured during certain activities (e.g., where different wearable devices 120 are capable of measuring at different frequencies), controller 110 may use wearable devices 120 to detect what activity user 130 is currently engaged in, and controller 110 may therein cause whichever wearable device 120 can measure at the desired frequency to measure a respective condition for the detected current activity.

In some examples, controller 110 may initialize this process by querying each of wearable devices 120 to identify internal settings and different options with which wearable devices 120 may measure conditions. Controller 110 may quantify and qualify the output from these settings, cross-referencing conditions data measured under different settings by different wearable devices 120 to determine respective characteristics of wearable devices 120.

As described above, controller 110 may be part of a computing device that includes a processor configured to execute instructions stored on a memory to execute the techniques described herein. For example, FIG. 2 is a conceptual box diagram of such computing system 200 of controller 110. While controller 110 is depicted as a single entity (e.g., within a single housing) for the purposes of illustration, in other examples, controller 110 may include two or more discrete physical systems (e.g., within two or more discrete housings). Controller 110 may include interfaces 210, processor 220, and memory 230. Controller 110 may include any number or amount of interface(s) 210, processor(s) 220, and/or memory(s) 230.

Controller 110 may include components that enable controller 110 to communicate with (e.g., send data to and receive and utilize data transmitted by) devices that are external to controller 110. For example, controller 110 may include interface 210 that is configured to enable controller 110 and components within controller 110 (e.g., such as processor 220) to communicate with entities external to controller 110. Specifically, interface 210 may be configured to enable components of controller 110 to communicate with wearable devices 120 or the like. Interface 210 may include one or more network interface cards, such as Ethernet cards and/or any other types of interface devices that can send and receive information. Any suitable number of interfaces may be used to perform the described functions according to particular needs.

As discussed herein, controller 110 may be configured to manage measurements gathered by wearable devices 120 regarding conditions related to users 130. Controller 110 may utilize processor 220 to thusly manage measurements. Processor 220 may include, for example, microprocessors, digital signal processors (DSPs), application specific integrated circuits (ASICs), field-programmable gate arrays (FPGAs), and/or equivalent discrete or integrated logic circuits. Two or more of processor 220 may be configured to work together to manage measurements accordingly.

Processor 220 may manage measurements of wearable devices 120 according to instructions 232 stored on memory 230 of controller 110. Memory 230 may include a computer-readable storage medium or computer-readable storage device. In some examples, memory 230 may include one or more of a short-term memory or a long-term memory. Memory 230 may include, for example, random access memories (RAM), dynamic random-access memories (DRAM), static random-access memories (SRAM), magnetic hard discs, optical discs, floppy discs, flash memories, forms of electrically programmable memories (EPROM), electrically erasable and programmable memories (EEPROM), or the like. In some examples, processor 220 may manage measurements as described herein according to instructions 232 of one or more applications (e.g., software applications) stored in memory 230 of controller 110.

In addition to instructions 232, in some examples gathered or predetermined data or techniques or the like as used by processor 220 to manage measurements as described herein may be stored within memory 230. For example, memory 230 may include information described above as controller 110 gathers from wearable devices 120. For example, as depicted in FIG. 2, memory 230 may include wearable device data 234, which includes measurement data 236 and characteristic data 238. Wearable device data 234 may include an identification of each of wearable devices 120, a list of various settings that may be used to measure conditions for the respective wearable device, and/or a list of conditions that each wearable device 120 can measure. Measurement data 236 may include all of the measurements gathered by respective wearable devices 120. Measurement device 236 may be stored in a format that cross-correlates each measurement to a timestamp and/or activity of the respective user. In this way, controller 110 may compare measurement data 236 measured at a given time by a respective wearable device 120 with measurement data 236 measured at the same time by another wearable device 120 as described herein. Characteristic data 238 may include data on respective wearable devices 120, such as a current battery level of the respective wearable device 120, an accuracy with which respective wearable devices 120 measure respective conditions, accuracy, frequencies with which respective wearable devices 120 may capture measurements, or the like.

Further, memory 230 may include threshold data 240. Threshold data 240 may include data on thresholds as to when controller 110 may switch between one determination and another. For example, threshold data 240 may detail a battery level at which controller 110 is to switch between wearable devices 120. For another example, threshold data 240 may include data on a threshold amount of accuracy with which each condition should be measured. For example, some conditions during some activities may be associated with a relatively lower accuracy level such that a specific number at a small granularity is relatively less important (e.g., a heart rate when user 130 is sleeping), while during other activities these same conditions are to be measured as accurately and as incrementally as possible (e.g., a heart rate when user 130 is exercising).

Memory 230 may further include machine learning techniques 242 that controller 110 may use to improve a process of managing condition measurement over time, as discussed herein. Machine learning techniques 242 can comprise algorithms or models that are generated by performing supervised, unsupervised, or semi-supervised training on a dataset, and subsequently applying the generated algorithm or model to manage condition measurement.

Machine learning techniques 242 can include, but are not limited to, decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques.

For example, machine learning techniques 242 can utilize one or more of the following example techniques: K-nearest neighbor (KNN), learning vector quantization (LVQ), self-organizing map (SOM), logistic regression, ordinary least squares regression (OLSR), linear regression, stepwise regression, multivariate adaptive regression spline (MARS), ridge regression, least absolute shrinkage and selection operator (LASSO), elastic net, least-angle regression (LARS), probabilistic classifier, naïve Bayes classifier, binary classifier, linear classifier, hierarchical classifier, canonical correlation analysis (CCA), factor analysis, independent component analysis (ICA), linear discriminant analysis (LDA), multidimensional scaling (MDS), non-negative metric factorization (NMF), partial least squares regression (PLSR), principal component analysis (PCA), principal component regression (PCR), Sammon mapping, t-distributed stochastic neighbor embedding (t-SNE), bootstrap aggregating, ensemble averaging, gradient boosted decision tree (GBRT), gradient boosting machine (GBM), inductive bias algorithms, Q-learning, state-action-reward-state-action (SARSA), temporal difference (TD) learning, apriori algorithms, equivalence class transformation (ECLAT) algorithms, Gaussian process regression, gene expression programming, group method of data handling (GMDH), inductive logic programming, instance-based learning, logistic model trees, information fuzzy networks (IFN), hidden Markov models, Gaussian naïve Bayes, multinomial naïve Bayes, averaged one-dependence estimators (AODE), Bayesian network (BN), classification and regression tree (CART), chi-squared automatic interaction detection (CHAID), expectation-maximization algorithm, feedforward neural networks, logic learning machine, self-organizing map, single-linkage clustering, fuzzy clustering, hierarchical clustering, Boltzmann machines, convolutional neural networks, recurrent neural networks, hierarchical temporal memory (HTM), and/or other machine learning algorithms.

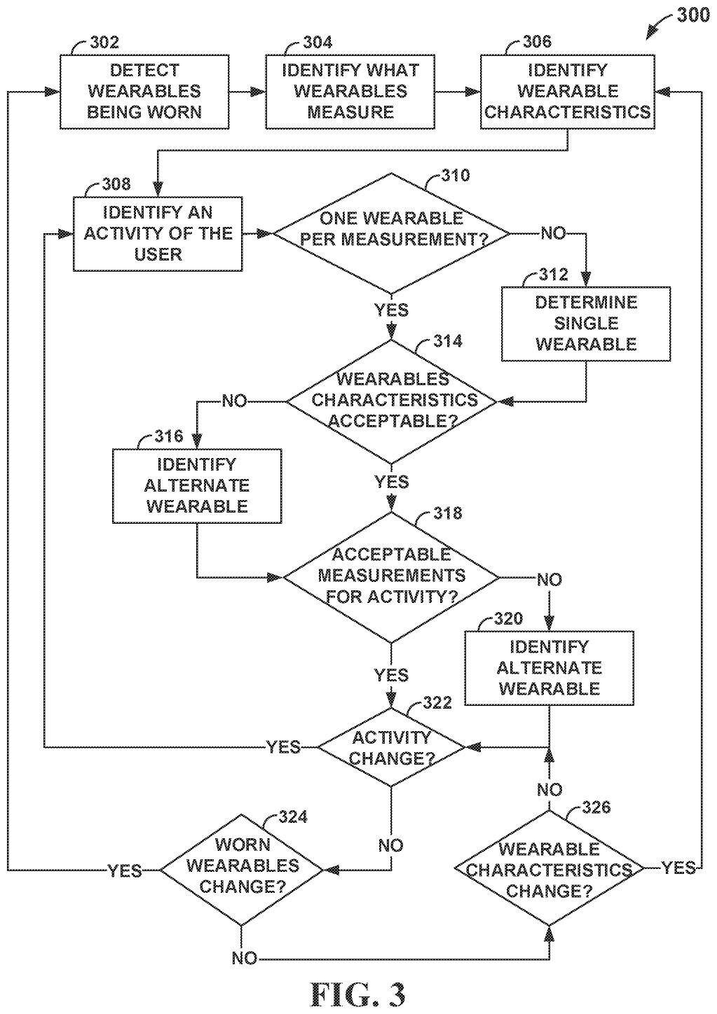

Using these components, controller 110 may manage measurements of conditions of a user for a plurality of wearable devices 120 as discussed herein. For example, controller 110 may manage condition measurement according to flowchart 300 depicted in FIG. 3. Flowchart 300 of FIG. 3 is discussed with relation to FIG. 1 for purposes of illustration, though it is to be understood that other systems may be used to execute flowchart 300 of FIG. 3 in other examples. Further, in some examples controller 110 may execute a different method than flowchart 300 of FIG. 3, or controller 110 may execute a similar method with more or less steps in a different order, or the like.

Controller 110 detects what wearable devices 120 are worn (302). Controller 110 may detect this as a number of wearable devices 120 that are synched to a master device (e.g., a cell phone of user 130), or are turned on, or are moving, or the like. Controller 110 may then identify what each of wearable devices 120 measure (304). This includes identifying duplicates, which includes two or more wearable devices 120 that are configured to measure a same condition of user 130. Controller 110 may identify characteristics of each of wearable devices 120 (306). Characteristics may include battery levels, efficiencies, accuracies, or the like. In some examples, controller 110 may identify that some characteristics of some wearable devices 120 are dependent on different activities.

Controller 110 may identify an activity of user 130 (308). Controller 110 may identify an activity of user 130 from gathered measurements. For example, if measurements include a high heart rate condition and a quick footstep condition, controller 110 may identify that user 130 is working out. Controller 110 may also identify an activity of user 130 from a location of user 130 (e.g., where GPS coordinates indicate that user 130 is at work), a schedule of user 130 (e.g., where a calendar accessible to controller 110 indicates that user 130 is an appointment), visual or audible information gathered from wearable devices 120 (e.g., where a video feed from a camera of one of wearable devices 120 includes images from a fitness class), or the like.

Controller 110 may evaluate whether or not more than one wearable device 120 is measuring each condition (310). If controller 110 determines that more than one of wearable devices 120 is measuring the condition, controller 10 may determine a single wearable device 120 to measure the condition as described herein. In the absence of other factors, controller 110 may determine which wearable devices 120 is to measure the condition at random. If each measurement is only gathered by a single wearable device 120 (and/or after each measurement is narrowed to a single wearable device 120 at 312), controller 110 may determine if the characteristics of each of wearable devices 120 are appropriate for those measurements allocated to them (314). For example, controller 110 may evaluate whether a battery level of one of wearable devices 120 is too low to measure a certain resource-intensive condition. If controller 110 determines that the characteristics of any wearable device 120 is not acceptable, controller 110 may determine an alternate wearable device 120 with more appropriate characteristics (316). For example, controller 110 may cause a wearable device 120 with more battery life (and/or with a more efficient measuring mechanism) to measure that condition.

If controller 110 determines that characteristics are acceptable (and/or after controller 110 causes alternate wearable devices with acceptable characteristics to measure the condition at 316), controller 110 may verify that measurements as gathered by the determined wearable devices 120 are acceptable for the detected activity (318). For example, controller 110 may analyze whether or not a frequency, reliability, accuracy, or the like of each of wearable devices 120 is acceptable for the identified activity. If controller 110 determines that the selected wearable devices 120 are not configured to measure their conditions according to acceptable thresholds, controller 110 may identify alternate wearable devices 120 that are able to measure these conditions according to the predetermined thresholds (320). In some examples, where none of worn wearable devices 120 can measure a condition according to a predetermined threshold, controller 110 may cause whichever wearable device 120 is closest to satisfying the threshold to measure the condition.

In some examples, only certain conditions are measured for certain activities. For example, user 130 may determine that a heart rate of user 130 is only to be gathered while user 130 is working out. In such examples, if controller 110 detects that user 130 is not working out, then controller 110 may identify that no wearable devices 120 should be measuring this condition. As such, controller 110 may dynamically update which conditions the set of wearables devices 120 are measuring over time in response to different detected activities of user 130.

Once controller 110 determines that all wearable devices 120 are measuring the conditions in a manner that is acceptable for the activity, controller 110 may go into a monitoring loop. When in this monitoring loop, controller 110 may be monitoring a status of user 130 and wearable devices 120 to see if any changes warrant changing which wearables devices 120 are monitoring which conditions. By monitoring statuses of both user 130 and wearable devices 120, controller 110 may dynamically manage measurements of conditions of user 130 over time.

For example, controller 110 monitors for whether or not an activity of user 130 changes (322). Controller 110 may determine that an activity changes by detecting that a location of user 130 changes, measured conditions of user 130 change, an image or audio feed associated with user 130 indicate an activity change, or the like. If controller 110 determines that an activity does change, controller 110 may identify the new activity of user 130 (308) and continue flowchart 300 from there.

If controller 110 detect that the activity has remained the same, controller 110 may verify whether or not a set of worn wearable devices 120 changes (324). Controller 110 may determine that such a set changes as a result of one or more of wearable devices 120 turning on, turning off, staying still, start moving, connecting to a master device of user 130, or the like. If controller 110 detects that the set of wearable devices 120 changes (whether because a total number of wearable devices 120 increases, decreases, or stays the same overall), controller 110 may detect those wearable devices 120 that are worn (302) and continue flowchart 300.

If controller 110 does not detect a change in the set of worn wearable devices 120, controller 110 may verify whether or not there is a change in characteristics of wearable devices 120 (326). For example, controller 110 may detect whether or not there is a change in a battery level of one or more wearable devices 120 that is greater than a threshold change. For another example, controller 110 may detect whether or not there is a change of accuracy of measurements gathered by one or more wearable devices 120 (e.g., where heart rate measurements do not make sense, such that controller 110 determines that a heart rate monitor is no longer properly fitting against a chest of user 130). In such examples where controller 110 detects a change in characteristics that surpasses a threshold, controller 110 may identify current characteristics of all wearable devices 120 (306), and therein continue flowchart 300 from there.

Otherwise, as described herein, controller 110 may substantially continue cycling through a monitoring cycle (322, 324, 326) in which controller 110 dynamically evaluates (and therein moves through flowchart 300 to update) how wearable devices 120 can and should monitor user 130. Controller 110 may cycle through this monitoring cycle (322, 324, 326) at a predetermined pace, such as going through this cycle once every minute. In some examples, a pace may depend upon the activity, where controller 110 cycles relatively faster during some activities (e.g., during user 130 exercise) and cycles relatively slower during other activities (e.g., during user 130 sleep).

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-situation data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method comprising:
determining that first and second wearable devices are both worn by a user during a first activity and that both of the first and second wearable devices are configured to measure a same condition relating to the user;
determining, based on how characteristics of the first and second wearable devices relate to the first activity, to measure the same condition with only the first wearable device;
determining that the first and second wearable devices are both worn by the user during a second activity; and
determining, based on how the characteristics of the first and second wearable devices relate to the second activity, to measure the same condition with only the second wearable device.

2. The computer-implemented method of claim 1, wherein the characteristics include respective battery levels.

3. The computer-implemented method of claim 1, wherein the characteristics include respective accuracy levels with which the first and second wearable devices measure the same condition during the first and second activities.

4. The computer-implemented method of claim 1, wherein the characteristics include respective frequency levels with which the first and second wearable devices measure the same condition during the first and second activities.

5. The computer-implemented method of claim 1, wherein the characteristics include respective efficiency levels with which the first and second wearable devices measure the same condition during the first and second activities.

6. The computer-implemented method of claim 1, further comprising:
determining that the second wearable device and a third wearable device are both worn by the user during a subsequent session of the first activity and are both configured to measure the same condition relating to the user;
determining that the first wearable device is not worn by the user during the subsequent session of the first activity; and
determining, based on how characteristics of the second and third wearable devices relate to the first activity, to measure the same condition with only the second wearable device.

7. The computer-implemented method of claim 1, further comprising:
causing both the first and second wearable devices to simultaneously measure the same condition during an initializing session of the first activity; and
comparing the same condition as measured by both the first and second wearable devices to identify the characteristics of the first and second wearable devices.

8. A system comprising:
a processor; and
a memory in communication with the processor, the memory containing instructions that, when executed by the processor, cause the processor to:
determine that first and second wearable devices are both worn by a user during a first activity and that both the first and second wearable devices are configured to measure a same condition relating to the user;
determine, based on how characteristics of the first and second wearable devices relate to the first activity, to measure the same condition with only the first wearable device;
determine that the first and second wearable devices are both worn by the user during a second activity; and
determine, based on how the characteristics of the first and second wearable devices relate to the second activity, to measure the same condition with only the second wearable device.

9. The system of claim 8, wherein the characteristics include respective battery levels.

10. The system of claim 8, wherein the characteristics include respective accuracy levels with which the first and second wearable devices measure the same condition during the first and second activities.

11. The system of claim 8, wherein the characteristics include respective frequency levels with which the first and second wearable devices measure the same condition during the first and second activities.

12. The system of claim 8, wherein the characteristics include respective efficiency levels with which the first and second wearable devices measure the same condition during the first and second activities.

13. The system of claim 8, the memory containing additional instructions that, when executed by the processor, cause the processor to:
determine that the second wearable device and a third wearable device are both worn by the user during a subsequent session of the first activity and are both configured to measure the same condition relating to the user;
determine that the first wearable device is not worn by the user during the subsequent session of the first activity; and
determine, based on how characteristics of the second and third wearable devices relate to the first activity, to measure the same condition with only the second wearable device.

14. The system of claim 8, the memory containing additional instructions that, when executed by the processor, cause the processor to:
cause both the first and second wearable devices to simultaneously measure the same condition during an initializing session of the first activity; and
compare the same condition as measured by both the first and second wearable devices to identify the characteristics of the first and second wearable devices.

15. A computer program product, the computer program product comprising a computer readable storage medium having program instructions embodied therewith, the program instructions executable by a computer to cause the computer to:
determine that first and second wearable devices are both worn by a user during a first activity and that both the first and second wearable devices are configured to measure a same condition relating to the user;
determine, based on how characteristics of the first and second wearable devices relate to the first activity, to measure the same condition with only the first wearable device;
determine that the first and second wearable devices are both worn by the user during a second activity; and
determine, based on how the characteristics of the first and second wearable devices relate to the second activity, to measure the same condition with only the second wearable device.

16. The computer program product of claim 15, wherein the characteristics include respective accuracy levels with which the first and second wearable devices measure the same condition during the first and second activities.

17. The computer program product of claim 15, wherein the characteristics include respective frequency levels with which the first and second wearable devices measure the same condition during the first and second activities.

18. The computer program product of claim 15, wherein the characteristics include respective efficiency levels with which the first and second wearable devices measure the same condition during the first and second activities.

19. The computer program product of claim 15, the computer readable storage medium containing additional program instructions that, when executed by the computer, cause the computer to:
determine that the second wearable device and a third wearable device are both worn by the user during a subsequent session of the first activity and are both configured to measure the same condition relating to the user;
determine that the first wearable device is not worn by the user during the subsequent session of the first activity; and
determine, based on how characteristics of the second and third wearable devices relate to the first activity, to measure the same condition with only the second wearable device.

20. The computer program product of claim 15, the computer readable storage medium containing additional program instructions that, when executed by the computer, cause the computer to:
cause both the first and second wearable devices to simultaneously measure the same condition during an initializing session of the first activity; and
compare the same condition as measured by both the first and second wearable devices to identify the characteristics of the first and second wearable devices.
